(19) United States
(12) Patent Application Publication
(10) Pub. No.: US 2020/0337849 A1
(43) Pub. Date: Oct. 29, 2020

(54) HIP IMPLANT WITH ELASTIC RETENTION DEVICE

(71) Applicants: Joella Marie Knopf, Denver, NC (US); Samuel Reed Fox, Winterville, NC (US); Nathan Lawrence Lehr, Raleigh, NC (US); Joshua Landon Brazda, Morehead City, NC (US); Amy Michelle Bandy, Raleigh, NC (US); Dong In Kim, Apex, NC (US); George Grant Lane, III, Bloomington, IN (US); Nicole Ann Spencer, Madison, WI (US); Claire Elizabeth Vallery, Madison, WI (US)

(72) Inventors: Joella Marie Knopf, Denver, NC (US); Samuel Reed Fox, Winterville, NC (US); Nathan Lawrence Lehr, Raleigh, NC (US); Joshua Landon Brazda, Morehead City, NC (US); Amy Michelle Bandy, Raleigh, NC (US); Dong In Kim, Apex, NC (US); George Grant Lane, III, Bloomington, IN (US); Nicole Ann Spencer, Madison, WI (US); Claire Elizabeth Vallery, Madison, WI (US)

(21) Appl. No.: 16/854,659

(22) Filed: Apr. 21, 2020

Related U.S. Application Data
(60) Provisional application No. 62/837,262, filed on Apr. 23, 2019.

(51) Int. Cl. A61F 2/32; A61F 2/34; A61F 2/36

(52) U.S. Cl. CPC A61F 2/32; A61F 2/34; A61F 2002/30092; A61F 2002/3241; A61F 2310/00011; A61F 2/3609

(57) ABSTRACT

Implant devices, systems, and methods are used to prevent post-surgical dislocation of a surgically reconstructed ball-and-socket joint, while retaining the patient's range of motion. The implant device can have a cup having a substantially hemispherical inner contour; a head at least partially inserted within the cup, the head having an outer contour that is generally spherically-shaped, and an elastic retainer with an inner contour that is at least a cross-sectional segment of a spherical shape having substantially a same radius as the inner contour of the cup, the elastic retainer being attachable over an open end of the cup to retain the head within the cup and allowing the outer contour of the head to freely rotate and/or pivot against the inner contour of the cup over the predetermined range of motion of the ball-and-socket joint.

### RELATED APPLICATIONS

This application claims priority to U.S. Provisional Patent Application No. 62/837,262, filed on Apr. 23, 2019, the disclosure of which is incorporated by reference in its entirety.

### FIELD OF INVENTION

The subject matter disclosed herein relates generally to surgical implant devices, systems, and methods. More particularly, the subject matter disclosed herein relates to devices, systems, and methods for reconstructive surgical implantation of replacement joints for ball-and-socket joints.

### BACKGROUND

Total hip arthroplasty (hip replacement) procedures are extremely prevalent in the United States, however there is a large risk for dislocation following surgery. During a hip dislocation, the femoral stem impinges on the edge of the acetabular cup, causing the head of the femur to come out of the socket and into the surrounding tissue. Dislocations are painful and costly, as they generally require subsequent surgeries. The typical hip utilizes tendons and ligaments attached from the acetabular cup to the femoral head to prevent dislocations from occurring, however these important features may become at least temporarily compromised during surgery, due to the removal of the acetabular cup and femoral head.

Current standard implants provide no system of preventing dislocation other than relying on the remaining surrounding muscles and tissue for support. Other implants on the market have been shown to reduce the rate of dislocation, however have life spans less than about 10 years, requiring additional surgeries in patients who outlast their implant. Additionally, in patients at a much higher risk for dislocations, there are implants which provide a rigid retention system that prevents dislocations but extremely limits the patient's range of motion.

Therefore, a need exists in the field for a novel hip implant system that provides a decreased risk of hip dislocation, maintains patient range of motion, and has a lifespan greater than 10 years.

### SUMMARY

According to one aspect, an implant system for reconstructive surgery of a ball-and-socket joint is provided, the implant system comprising: a cup having a substantially hemispherical inner contour; a head configured for at least partial insertion within the cup, the head having an outer contour that is spherically-shaped, at least over portions of the head that are capable of contacting the inner contour of the cup over a predetermined range of motion of the ball-and-socket joint; and an elastic retainer comprising an inner contour that is at least a cross-sectional segment of a spherical shape having substantially a same radius as the inner contour of the cup, wherein the elastic retainer is configured for attachment over an open end of the cup to partially cover and retain the head within the cup, and wherein, when securing the head within the cup, the elastic retainer is configured to allow the outer contour of the head to freely rotate and/or pivot against the inner contour of the cup over the predetermined range of motion of the ball-and-socket joint; wherein the elastic retainer is configured to prevent post-surgical dislocation of the head from the cup.

In some embodiments of the implant system, the elastic retainer comprises an elastic material having an elasticity and tensile strength that is sufficient to provide dynamic support to the head and cup.

In some embodiments of the implant system, the elastic retainer comprises a high-durometer elastomeric material.

In some embodiments of the implant system, the inner contour of the elastic retainer forms a lip at an upper surface of the elastic retainer, the lip having a narrower diameter than a diameter of the head for retaining the head within the cup.

In some embodiments of the implant system, the elastic component has an exterior channel formed circumferentially about and adjacent to the upper surface of the elastic retainer, wherein the exterior channel is configured to retain a locking ring therein.

In some embodiments of the implant system, the locking ring is positioned within the exterior channel of the elastic retainer and is configured to resist radial expansion of the elastic retainer to resist a dislocation of the head from the cup.

In some embodiments of the implant system, the locking ring comprises a first end and a second end, the second end having a recess formed therein, wherein the first end is configured for insertion within the recess of the second end to secure the first and second ends of the locking ring together in a substantially continuous annular shape.

In some embodiments of the implant system, each of the first and second ends comprise an internal cavity, wherein one or more shape-memory wires is positioned to extend between and into the cavity of the first end and the cavity of the second end, and wherein the one or more shape-memory wires are configured to change a shape thereof based on a temperature of the one or more shape-memory wires.

In some embodiments of the implant system, the one or more shape-memory wires comprise Nitinol.

In some embodiments of the implant system, the one or more shape-memory wires are substantially straight at a first temperature, wherein a retention feature is formed within each cavity of the first and second ends, and wherein the one or more shape-memory wires are configured to engage with the retention features of each cavity at a second temperature to pull the first end of the locking ring within the second end of the locking ring to substantially entirely fill the recess of the second end of the locking ring.

In some embodiments of the implant system, the inner contour of the elastic component comprises a low friction bearing surface.

In some embodiments of the implant system, a lower surface of the elastic retainer is configured to be fixed coincidently and concentrically over an upper, open surface of the cup, the elastic retainer having a substantially similar diameter to a diameter of the cup at the upper, open surface of the cup.

In some embodiments of the implant system, the cup comprises an inner liner that is concentrically attached within the cup, the inner liner comprising an upper annular flange extending from an upper surface of the cup, wherein the elastic retainer comprises an annular channel having a cross-sectional shape that is substantially similar to a cross-sectional shape of the upper annular flange, and wherein the upper annular flange of the inner liner is inserted within the annular channel of the elastic retainer to affix the elastic retainer to the cup.

In some embodiments of the implant system, the elastic retainer is molded over the upper annular flange of the inner liner, such that the elastic retainer and the inner liner are of a substantially unitary, or monolithic, construction.

In some embodiments of the implant system, the upper annular flange comprises holes formed through a thickness thereof in the radial direction, such that a material of the elastic retainer solidifies within the holes to secure the elastic retainer to the inner liner.

In some embodiments of the implant system, the elastic retainer comprises holes in one or more sides of the elastic retainer that allow, when the head receives a force in a direction of dislocation, for the elastic retainer to be compressed and/or deformed by reducing a size of the holes, while maintaining sufficient tensile strength of the elastic retainer to retain the head within the cup.

In some embodiments of the implant system, the head and/or cup comprise a metal or metal alloy.

According to another aspect, an implant system for reconstructive surgery of a hip joint is disclosed, the implant system comprising: a cup fixed to the acetabulum of the pelvis, wherein the cup comprises a substantially hemispherical inner contour with a hemispherical lining of ultra high molecular weight polyethylene nested concentrically within the cup; a stem fixed within a femur; a head attached to the stem, wherein the head has an outer contour that is spherically-shaped, at least over portions of the head that are capable of contacting the inner contour of the cup over a predetermined range of motion of the hip joint, and is freely rotatable and/or pivotable within the cup; and an elastic retainer comprising an inner contour that is at least a cross-sectional segment of a spherical shape having substantially a same radius as the inner contour of the cup, wherein the elastic retainer is configured for attachment over an open end of the cup to partially cover and retain the head within the cup while the head is within the cup, and wherein, when securing the head within the cup, the elastic retainer is configured to allow the outer contour of the head to freely rotate and/or pivot against the inner contour of the cup over the predetermined range of motion of the ball-and-socket joint; wherein the elastic retainer is configured to prevent post-surgical dislocation of the head from the cup.

In some embodiments of the implant system, the elastic retainer comprises an elastic material having an elasticity and tensile strength that is sufficient to provide dynamic support to the head and cup and/or wherein the elastic retainer comprises a high-durometer elastomeric material.

In some embodiments of the implant system, the inner contour of the elastic retainer forms a lip at an upper surface of the elastic retainer, the lip having a narrower diameter than a diameter of the head for retaining the head within the cup.

In some embodiments of the implant system, the elastic component has an exterior channel formed circumferentially about and adjacent to the upper surface of the elastic retainer, wherein the exterior channel is configured to retain a locking ring therein.

In some embodiments of the implant system, the locking ring is positioned within the exterior channel of the elastic retainer and is configured to resist radial expansion of the elastic retainer to resist a dislocation of the head from the cup.

In some embodiments of the implant system, the inner contour of the elastic component comprises a low friction bearing surface.

In some embodiments of the implant system, a lower surface of the elastic retainer is configured to be fixed coincidently and concentrically over an upper, open surface of the cup, the elastic retainer having a substantially similar diameter to a diameter of the cup at the upper, open surface of the cup.

In some embodiments of the implant system, the elastic retainer comprises holes in one or more sides of the elastic retainer that allow, when the head receives a force in a direction of dislocation, for the elastic retainer to be compressed and/or deformed by reducing a size of the holes, while maintaining sufficient tensile strength of the elastic retainer to retain the head within the cup.

In some embodiments of the implant system, the head, stem, and/or cup comprise a metal or metal alloy.

According to still another aspect, a method of surgically reconstructing a ball-and-socket joint in a subject is provided, the method comprising: providing the subject in need of surgical reconstruction of the ball-and-socket joint; providing an implant device for surgically reconstructing the ball-and-socket joint, the implant device comprising: a cup having a substantially hemispherical inner contour, a head having an outer contour that is spherically-shaped, at least over portions of the head that are capable of contacting the inner contour of the cup over a predetermined range of motion of the ball-and-socket joint when implanted within the subject, and an elastic retainer comprising an inner contour that is at least a cross-sectional segment of a spherical shape having substantially a same radius as the inner contour of the cup; the method comprising attaching the cup to or within a first anatomical structure of the subject, the first anatomical structure corresponding to a socket portion of the ball-and-socket joint; inserting the head, at least partially, within the cup; attaching the elastic retainer over an open end of the cup to partially cover and retain the head within the cup, thereby securing the head within the cup, wherein the elastic retainer allows the outer contour of the head to freely rotate and/or pivot against the inner contour of the cup over the predetermined range of motion of the ball-and-socket joint; attaching a stem to or within a second anatomical structure of the subject, the second anatomical structure corresponding to a ball portion of the ball-and-socket joint; fixedly attaching the head to the stem, so that the head is rigidly attached to the stem to prevent relative movements between the head and the stem; and resisting, when a force is received in a direction of dislocation, a dislocation of the head from the cup after surgery.

In some embodiments of the method, the first anatomical structure is an acetabulum of a pelvis of the subject and the second anatomical structure is a femur of the subject.

In some embodiments of the method, the subject is a human.

In some embodiments of the implant device, the elastic retainer comprises an elastic material having an elasticity and tensile strength that is sufficient to provide dynamic support to the head and cup.

In some embodiments of the method, the elastic retainer comprises a high-durometer elastomeric material.

In some embodiments of the method, the inner contour of the elastic retainer forms a lip at an upper surface of the elastic retainer, the lip having a narrower diameter than a diameter of the head for retaining the head within the cup.

In some embodiments of the method, the elastic component has an exterior channel formed circumferentially about and adjacent to the upper surface of the elastic retainer to retain a locking ring therein.

In some embodiments, the method comprises securing a locking ring within the exterior channel of the elastic retainer to resist radial expansion of the elastic retainer, thereby also resisting a dislocation of the head from the cup.

In some embodiments of the method, the locking ring comprises a first end and a second end, the second end having a recess formed therein; the method comprising inserting the first end within the recess of the second end to secure the first and second ends of the locking ring together in a substantially continuous annular shape.

In some embodiments of the method, each of the first and second ends comprise an internal cavity, wherein one or more shape-memory wires is positioned to extend between and into the cavity of the first end and the cavity of the second end, and wherein the one or more shape-memory wires are configured to change a shape thereof based on a temperature of the one or more shape-memory wires.

In some embodiments of the method, the one or more shape-memory wires comprise Nitinol.

In some embodiments of the method, the one or more shape-memory wires are substantially straight at a first temperature, wherein a retention feature is formed within each cavity of the first and second ends, and wherein the one or more shape-memory wires are configured to engage with the retention features of each cavity at a second temperature to pull the first end of the locking ring within the second end of the locking ring to substantially entirely fill the recess of the second end of the locking ring.

In some embodiments of the method, the inner contour of the elastic component comprises a low friction bearing surface.

In some embodiments, the method comprises fixing a lower surface of the elastic retainer coincidently and concentrically over an upper, open surface of the cup, the elastic retainer having a substantially similar diameter to a diameter of the cup at the upper, open surface of the cup.

In some embodiments of the method, the cup comprises an inner liner that is concentrically attached within the cup, the inner liner comprising an upper annular flange extending from an upper surface of the cup; the elastic retainer comprises an annular channel having a cross-sectional shape that is substantially similar to a cross-sectional shape of the upper annular flange; and the upper annular flange of the inner liner is inserted within the annular channel of the elastic retainer to affix the elastic retainer to the cup.

In some embodiments, the method comprises molding the elastic retainer over the upper annular flange of the inner liner, such that the elastic retainer and the inner liner are of a substantially unitary, or monolithic, construction.

In some embodiments of the method, the upper annular flange comprises holes formed through a thickness thereof in the radial direction, such that a material of the elastic retainer solidifies within the holes to secure the elastic retainer to the inner liner.

In some embodiments of the method, the elastic retainer comprises holes in one or more sides of the elastic retainer that allow, when the head receives a force in a direction of dislocation, for the elastic retainer to be compressed and/or deformed by reducing a size of the holes, while maintaining sufficient tensile strength of the elastic retainer to retain the head within the cup.

In some embodiments of the method, the head and/or cup comprise a metal or metal alloy.

### BRIEF DESCRIPTION OF THE DRAWINGS

FIG. 1 is an exploded view of an example embodiment of an implant system.

FIG. 2 is a side view of the implant system of FIG. 1.

FIG. 3 is a side perspective view of a proof-of-concept prototype of the example embodiment of the implant system of FIG. 1.

FIG. 4 is a side view of another example embodiment of an implant system.

FIG. 5 is an exploded side view of the implant system of FIG. 4.

FIG. 6A is an isolated exploded sectional view of the inner liner and the elastic retainer of the implant system shown in FIGS. 4 and 5.

FIG. 6B is an isolated sectional view of the inner liner and elastic retainer of the implant system, as shown in FIG. 6A, in an assembled state.

FIG. 7 is an isolated perspective view of the implant system of FIGS. 4-6, with the femoral stem being omitted for clarity.

FIGS. 8A-8C are schematic illustrations showing how the ring of the implant system is secured within the channel of the elastic retainer to resist dislocation of the head from within the cup.

### DETAILED DESCRIPTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “about,” when referring to a value or to an amount of a composition, mass, weight, temperature, time, volume, concentration, percentage, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions. As used herein, the term “substantially,” when referring to a value, an activity, or to an amount of a composition, mass, weight, temperature, time, volume, concentration, percentage, etc., is meant to encompass variations of in some embodiments ±40%, in some embodiments ±30%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions. As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole. As used herein, the phrase “consisting essentially of” limits the scope of a claim to the specified materials or steps, plus those that do not materially affect the basic and novel characteristic(s) of the claimed subject matter. With respect to the terms “comprising”, “consisting of”, and “consisting essentially of”, where one of these three terms is used herein, the presently disclosed and claimed subject matter can include the use of either of the other two terms.

The term “subject,” as used herein, generally refers to a mammal. Typically, the subject is a human. However, the term embraces other species, e.g., pigs, mice, rats, dogs, cats, or other primates. In certain embodiments, the subject is an experimental subject such as a mouse or rat. The subject may be a male or female. The subject may be an infant, a toddler, a child, a young adult, an adult or a geriatric. A subject under the care of a physician or other health care provider may be referred to as a “patient”. A “subject” of diagnosis or treatment is an animal, including a human. It also includes pets and livestock. As used herein, a “subject in need thereof” is a patient, animal, mammal, or human, who will benefit from the method of the presently disclosed subject matter.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

New implant systems with elastic retention are discussed herein. In the example embodiments described hereinbelow for purposes of explanation, the implant systems are used as femoral implants and numerous specific details are set forth in order to provide a thorough understanding of the example embodiments according to the present disclosure. It will be evident, however, to one skilled in the art that the present invention may be practiced without these specific details and, indeed, may be practiced in implant systems in other anatomical structures other than the human femur.

The present disclosure is to be considered as an example of the invention and is not intended to limit the invention to the specific embodiments illustrated by the figures or description below.

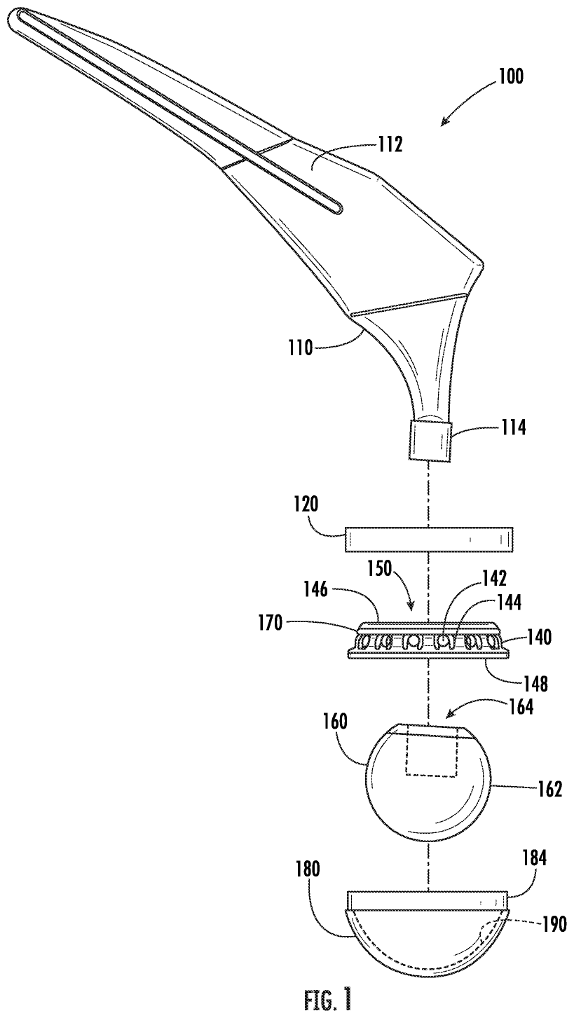

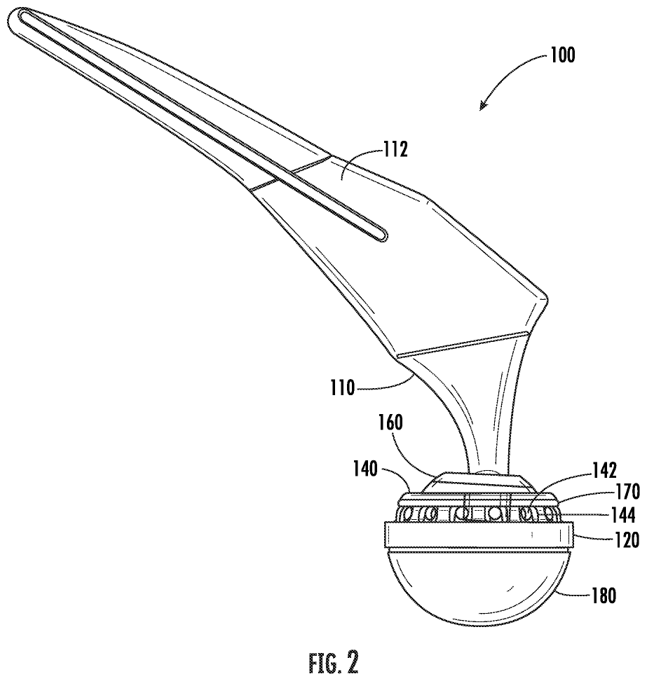

In FIGS. 1 and 2, various aspects of an implant system, generally designated 100, are shown. The implant system 100 shown is used for reconstructive surgery of a ball-and-socket joint in a patient and/or subject. The implant system 100 includes a femoral stem 110, a fastener 120, an elastic retainer 140 (e.g., having an annular, or ring, shape), a femoral head 160, and an acetabular cup 180. The femoral stem 110 has a body 112 that is inserted (e.g., longitudinally) and/or implanted within a bone of the subject in which the implant system 100 is to be inserted. At the end of the body 112, the femoral stem 110 has a coupler 114 that is able to be coupled to (e.g., by threadable engagement with a threaded surface of) the femoral head 160 during surgical implantation of the implant system 100. The coupler 114 is advantageously fixedly coupled within a recessed cavity 164 of the femoral head 160, such that the femoral stem 110 and the femoral head 160 are rotatably locked together to prevent relative movements therebetween after the surgical implantation of the implant system 100 within the subject.

In an assembled state of the implant system 100, the elastic retainer 140 is attached to the acetabular cup 180 using the fastener 120, thereby retaining the femoral head 160 at least partially within the acetabular cup 180. While the interface by which the fastener 120 is secured to the acetabular cup 180 to hold the elastic retainer 140 in position may be of any suitable type, in the example embodiment shown, the acetabular cup 180 has an annular flange portion 184 that is threaded on an outer circumferential surface thereof and the fastener 120 has an inner surface that has threads complementary to (e.g., the same size and pitch) the threads of the flange portion 184, such that the fastener 120 can be threadably engaged over the flange portion 184 after the femoral head 160 and the elastic retainer 140 are positioned within the acetabular flange 180 to pivotably secure the elastic retainer 140 and the femoral head 160 within the acetabular cup 180. Once the femoral head 160 is in the assembled position, retained within the acetabular cup 180 by the elastic retainer 140, the femoral stem 120 is attached to the femoral head 160. The elastic retainer 140 in the example embodiment disclosed herein comprises a high durometer elastomeric material (e.g., silicone, urethane, and/or any elastomeric material having a durometer greater than or equal to about Shore 30A), but may be made out of any suitable material with sufficient elasticity to provide dynamic support and tensile strength to the implant system 100 and is not limited thereto.

When the implant system 100 receives a force in the direction of dislocation (e.g., in the direction that would otherwise dislodge the femoral head 160 from the acetabular cup 180), the femoral stem 120 rotates in the ball-and-cup socket defined between the acetabular cup 180 and impinges on the elastic retainer 140, when the elastic retainer 140 is attached to the acetabular cup 180 by the fastener 120, resulting in the elastic retainer 140 being in compression (e.g., compressed in a radial direction) due to the dislocating movement of femoral stem 110. When the implant system 100 receives a force in the direction of impingement (e.g., in the direction that would not cause the femoral head 160 to pull away from the acetabular cup 180), the femoral head 160 is pressed against the inner surface of the acetabular cup 180, which can cause the elastic retainer 140 to be in tension. The elastic retainer 140, the femoral head 160, and the acetabular cup 180 are formed to allow a predetermined range of motion (e.g., pivoting and/or rotating) corresponding to a range of motion of the anatomical joint being replaced by the implant system 100.

In the example embodiment shown, the elastic retainer 140 comprises holes, openings, apertures or the like, but which will hereinafter be referred to as holes 142, in one or more sides of the elastic retainer 140 that allow for compression of the elastic retainer by deformation of the elastic retainer 140 that reduces the size of the holes 142 when the implant system 100 receives a force in the direction of dislocation. The holes 142 may be formed in the outer circumferential surface, the inner circumferential surface, or both the outer and inner circumferential surfaces of the elastic retainer 140, and may pass through all or only a portion of the thickness of the elastic retainer 140 in the radial direction of the elastic retainer 140. Holes may also, or alternatively, be formed in the upper and/or lower surfaces 146, 148. The remaining material of the elastic retainer 140 is designed to ensure that the elastic retainer 140 is able to provide the necessary strength in tension/compression. Vertically-aligned walls 144 may be provided around the holes 140 in the direction between the upper and/or lower surfaces 146, 148. The outer circumferential surface of the opening, generally designated 150, of the elastic retainer 140 is reinforced, for example, by a locking ring 170 made from a corrosion-resistant bio-compatible material (e.g., Ti-6AI-4V) in the example embodiment shown, to prevent the elastic retainer 140 from rupturing, or otherwise being deformed to a sufficient degree to allow the femoral head 160 to become dislocated from the acetabular cup 180 due to hoop forces (e.g., that are generally radially-aligned, relative to the elastic retainer 140) that are caused by movements of the femoral head 160 relative to the acetabular cup 180.

Stated differently, the elastic retainer 140 can be formed to have an inner contour that is shaped in a portion of (e.g., a cross-sectional section of) a substantially circular or spherical shape. The elastic retainer 140 is configured to be fixed to, or otherwise attached to, a distal face of an acetabular cup 180, which is a generally hemispherically-shaped metallic shell element of a hip implant in the example embodiment shown.

The acetabular cup 180 has an inner liner 190 defining an inner contour that is complementary to (e.g., the same as) the outer contour 162 of the femoral head 160, to allow a substantially unimpeded (e.g., resisted by frictional contact forces, but not due to interferences between the contact surfaces of the femoral head 160 and the acetabular cup 180). The inner contour 184 of the acetabular cup 180 is substantially no more than a hemisphere to allow for insertion of the femoral head 160 within the acetabular cup 180 during the assembly of the implant system 100 during surgical implantation. In some embodiments, the acetabular cup 180 may extend beyond a hemispherical shape to retain the femoral head 160 at least temporarily within the acetabular cup 180 during surgical implantation. In such embodiments, the portions of the acetabular cup extending beyond a hemispherical shape may have slots formed therein, or be made from an elastic, deformable material to allow for radial deflection of the portions of the acetabular cup 180 that would otherwise prevent insertion of the femoral head 160 within the acetabular cup 180.

The generally hemispherical shape of the acetabular cup 180 is advantageous, in that it at least partially covers a femoral head 160, which has a generally spherical outer surface in at least portions of the femoral head 160 that will be in contact within the acetabular cup 180 as the femoral head 160 is pivoted and/or twisted therein after surgical implantation in a patient, thereby allowing full rotation of the femoral head 160, as well as the femoral stem 110 attached (e.g., threadably) thereto, relative to the acetabular cup 180. The implant system 100 is thereby advantageous over known implant devices and systems at least for the reason that implant system 100 is able to substantially increase post-surgical retention of (e.g., prevent dislocation of) a hip implant, thereby improving patient outcomes.

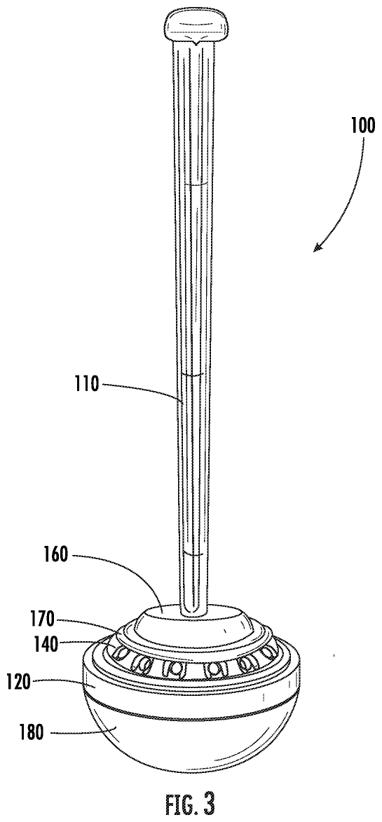

In the example embodiment shown in FIGS. 1-3, the implant system 100 is a hip implant system to be surgically implanted in a patient undergoing hip replacement surgery. The implant system 100 includes an acetabular cup 180, in the form of a hemispherical metallic shell element, which is fixed to the acetabulum of the pelvis during surgical implantation of the implant system 100. A hemispherical lining made of ultra high molecular weight polyethylene is provided along and/or over substantially all of the inner liner 182 of the acetabular cup 180. In the prototype shown in FIG. 3, the femoral stem 110 is in the shape of a metallic implant that could be fixed within a bore hole formed within the femur (e.g., along the length thereof) during surgical implantation. A femoral head 160 in the form of a substantially spherical head is threadably attached to the femoral stem 160, which fits concentrically within the acetabular cup 180, against the inner contour of the inner liner 190 thereof. The femoral head 160 can freely rotate and/or pivot within the acetabular cup 180 about all three axes (e.g., x, y, and z). An elastic retainer 140 is secured over the open end of the acetabular cup 180 to secure the elastic retainer 140 over the femoral head 160 and to retain the femoral head 160 within the acetabular cup 180 post-surgery.

In the prototype of the implant system 100 shown in FIG. 3, the acetabular cup 180 and the femoral head 160 were produced via additive manufacturing (e.g., by “3D printing”) from Formlabs® Durable Photopolymer Resin, and the elastic retainer 140 was produced via additive manufacturing from Formlabs® Flexible Photopolymer Resin. Each of the elastic retainer 140, the femoral head 160, and the acetabular cup 180 are formed using a Formlabs@ Form 2 additive manufacturing system, sometimes referred to as a 3D printer. In the prototype implant system 100, the elastic retainer 140 was affixed to the acetabular cup 180 using JB Weld® Original Cold-Weld Steel Reinforced Epoxy, rather than using the fastener 120 shown in the implant system of FIGS. 1 and 2. The elastic retainer 140 is reinforced by a metal locking ring 170, which was formed out of 19-gauge galvanized steel wire by wrapping the wire around the elastic retainer 140 and tightening the free ends of the wire with pliers until the femoral head 160 was held securely within the acetabular cup 180, but the femoral head 160 was still free to rotate within the acetabular cup 180. The femoral stem shown in the example embodiment of FIGS. 1 and 2 was replaced by a ⅜ inch bolt for use as a proof-of-concept implant system 100.

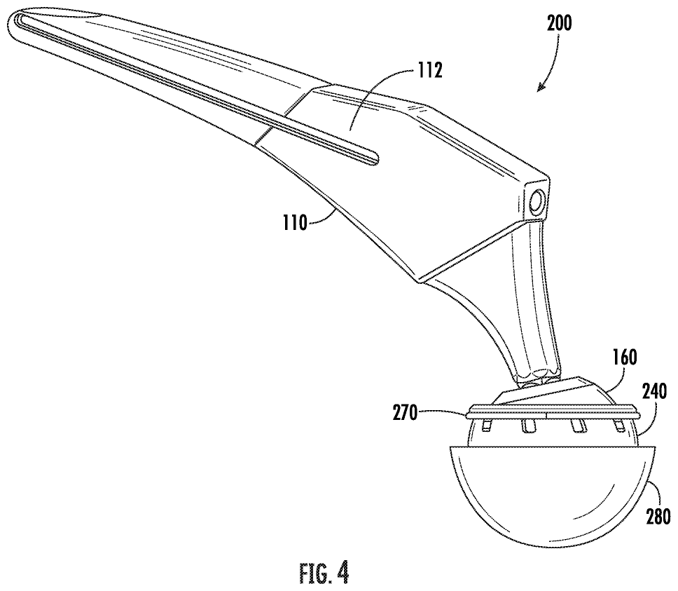

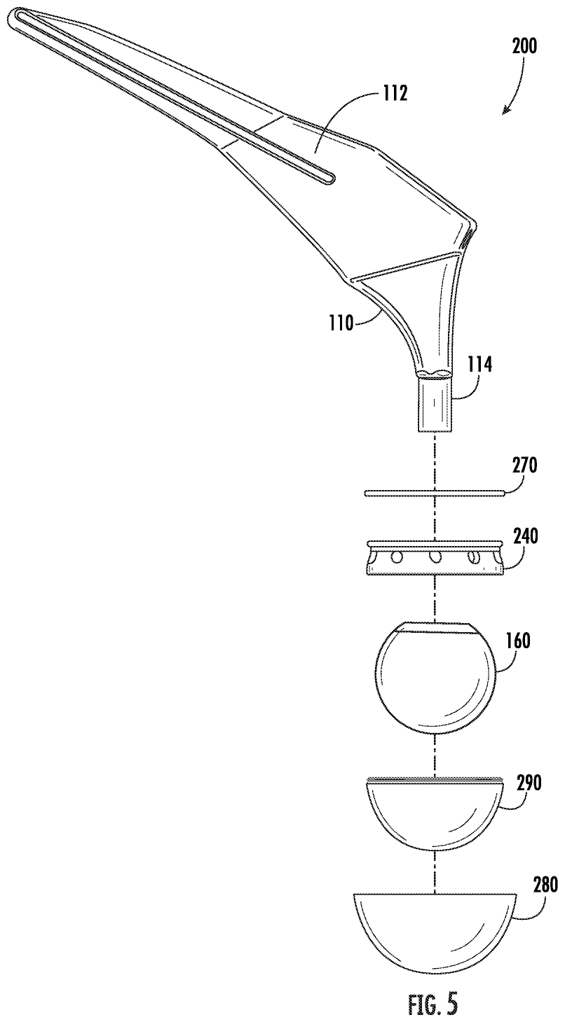

FIGS. 4-7 show aspects of a second example embodiment of an implant system, generally designated 200. While certain aspects of the implant system 200 differ from the implant system 100 of FIGS. 1-3, similarly numbered elements, features, and/or structures in implant system 200 are the same as in the implant system 100, or are at least substantially similar variants thereof. Such similar elements, features, and/or structures will not be discussed in great detail herein, but reference is made to the descriptions of such elements, features, and/or structures elsewhere herein in the descriptions of the example embodiment of the implant system 100 in FIGS. 1-3.

The implant system 200 has a femoral stem 110, which is lockingly inserted and secured within a femoral head 160. The femoral head 160 is pivotably and/or rotatably positioned within the acetabular cup 280, which can be substantially similar in construction to the acetabular cup 280 of the implant system 100. An inner liner 290 is fixedly attached to an inner surface of the acetabular cup 280, such that the inner liner 290 forms the contact surface for the femoral head. The inner liner 290 has an inner contour (e.g., radius) that is substantially similar to the outer contour (e.g., radius) of the femoral head 160, such that the inner liner 290 and the femoral head 160 have a distributed contact area to reduce frictional loads during use.

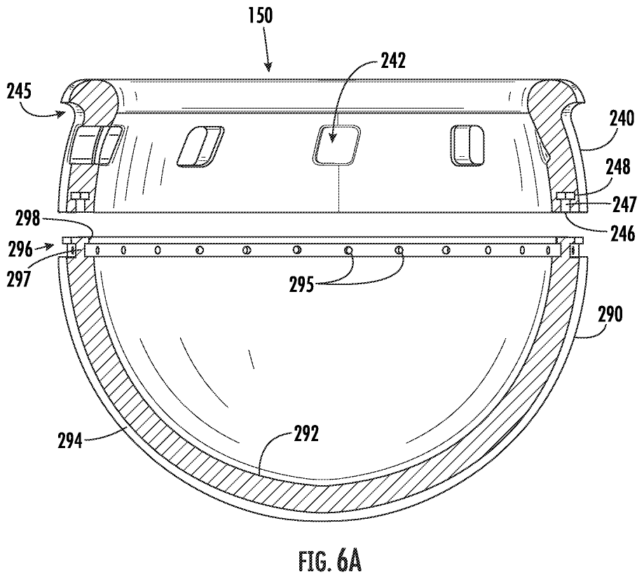

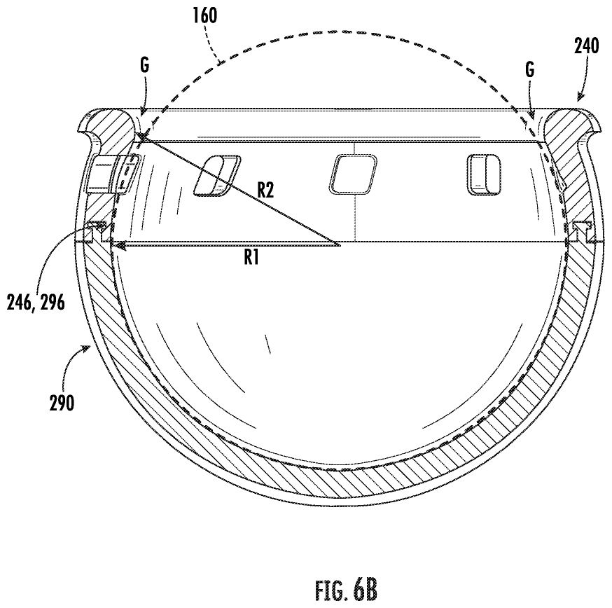

The implant system 200 includes an elastic retainer 240 that is secured to the upper surface of the acetabular cup 280, which has a hemispheric shape. Unlike in implant system 100, the elastic retainer 240 of implant system 200 is fixedly retained over the open end of the inner liner 290 of the acetabular cup 280. The elastic retainer 240 is formed with an annular channel 246, having a vertical section 247 extending substantially tangentially to the contour of the upper surface of the inner liner 290 and a branch section 248 that extends at a non-zero angle from the vertical section 247. In the embodiment shown, the branch section 248 is oriented at substantially a 90 degree angle from the vertical section 247, but the orientations of the vertical and branch sections 247, 248 are not limited thereto. In fact, the vertical section 247 can be inclined relative to the tangential arrangement described and shown in FIG. 6A and the branch section 248 can be at any suitable angle and can extend in one or more directions at an angle to the vertical section 247.

The inner liner 290 has an upper flange 296 formed therein. The upper flange 296 has a cross-sectional shape that is complementary to (e.g., capable of fitting within) the cross-sectional shape of the annular channel 246 of the elastic retainer 240. As shown, the upper flange has a vertical section 297 that corresponds to (e.g., is the same as, or is substantially similar to) the shape and dimensions of the vertical section 247 of the annular channel 246 of the elastic retainer 240 and a branch section 298 that corresponds to (e.g., is the same as, or is substantially similar to) the shape and dimensions of the branch section 248 of the annular channel 246 of the elastic retainer 240. In some embodiments, the elastic retainer 240 is formed via an overmolding process (e.g., by injection molding the elastic retainer 240 over at least the upper flange 296 of the inner liner 290) over the upper flange 296 of the inner liner 290 after the inner liner 290 has been formed, such that the elastic retainer 240 and the inner liner 290, along with the acetabular cup 280 in some embodiments, are formed from different or the same materials, but have a unitary, or monolithic structure.

The upper flange 296 can be shaped, and the elastic retainer 240 can be made of a sufficiently high durometer material, that elastic retainer 240 is substantially locked and/or rigidly attached over the upper surface of the inner liner 290, such that the elastic retainer 240 cannot be nondestructively removed from the inner liner 290. In the embodiment shown, the vertical section 297 of the upper flange 296 has one or more (e.g., advantageously, a plurality of) holes 295 formed through at least a portion of the thickness thereof, in the radial direction as shown in FIG. 6A. As shown, the holes 295 pass through the entire thickness of the upper flange 296 and the elastic retainer 240 is formed such that the material from which the elastic retainer 240 is formed passes through, and is formed within, each of the holes 295 during the overmolding process, thereby lockingly attaching the elastic retainer 240 to the inner liner at both the holes 295 and the upper flange 296 of the inner liner 290. As such, the annular channel 246 is advantageously formed during the overmolding process by the upper flange 296 of the inner liner 290.

The elastic retainer 240 has holes 242 formed therethrough which are substantially similar in function and/or form to the holes 142 of the elastic retainer 140 of the implant system 100, allowing for enhanced compression capabilities of the elastic retainer 240. As is shown in FIG. 6B, the elastic retainer 240 has a generally domed internal surface, such that the first radius R1 of the elastic retainer 240, as measured from the center point of the femoral head 160 to the inner surface of the elastic retainer 240 in the plane defined by the upper surface of the inner liner 290, is not as long as (e.g., is shorter than) a second radius R2, as measured from the center point of the femoral head 160 to the radial edge of the upper opening 150 of the elastic retainer 240.

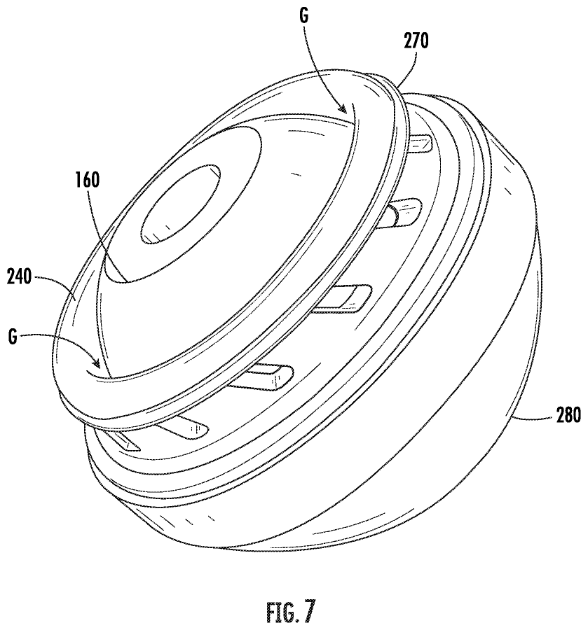

This domed, or elongated, profile of the inner contour of the elastic retainer 240 is advantageous because, during normal use of the implant system 100 as replacement for a hip joint, the femoral head 160 will not be in contact with the inner surface or contour of the elastic retainer 240 unless a force input is received which would cause a movement of the femoral head 160 in a direction away from the inner liner 290, in the direction of dislocation. Due to this domed, or elongated, profile of the inner contour of the elastic retainer 240, there is a gap G present between the outer surface of the femoral head 160 and the inner surface or contour of the elastic retainer 240 during typical use (e.g., in the absence of force inputs in the direction of dislocation). These gaps G between the femoral head 160 and the elastic retainer 240 can be seen in FIG. 7 and allow for subluxation of the femoral head 160 within the acetabular cup 280, thereby providing improved post-operative range of motion for subjects than in known hip implant devices and/or systems, while still resisting and/or preventing post-operative dislocation of the hip joint. In addition to the improved range of motion, the gap G is also advantageous for decreasing frictional wear of the components of the implant system 200, principally the femoral head 160 and/or any surfaces contacted by the femoral head 160. By virtue of the presence of the gap G, the elastic retainer 240 will only physically contact the femoral head 160 when the femoral head 160 is at risk being dislocated from the acetabular cup 280. At other times (e.g., when no force in the direction of dislocation is received at the femoral head 160), there will be no contact or frictional abrasion (e.g., rubbing) between the femoral head 160 and the elastic retainer 240, thereby advantageously increasing the usable life of the implant system 200.

Therefore, during surgical implantation of the implant system 200, the acetabular cup 280, along with the inner liner 290 fixedly attached concentrically therein, as well as the elastic retainer 240 rigidly and fixedly attached to the upper surface of the inner liner 290, is surgically implanted to the acetabulum of the pelvis of a subject. Once the acetabular cup 280 is secured at the surgical site, the femoral head 160 is inserted through the opening 150 of the elastic retainer 240, for example, by radially stretching the opening 150 of the elastic retainer 240 to allow the femoral head 160 to pass therethrough and be seated, or positioned, concentrically within the acetabular cup 280, resting against the inner surface thereof. After the insertion of the femoral head 160 into the acetabular cup 280, a locking ring 270 is secured within an external channel 245 formed radially about the outer circumference of the elastic retainer 240, for example, at or adjacent to the upper surface of the elastic retainer 240 where the opening 150 is formed. The channel 245 is recessed within the locking ring and has a lip about the upper surface to prevent the locking ring from becoming dislodged from the channel 245.

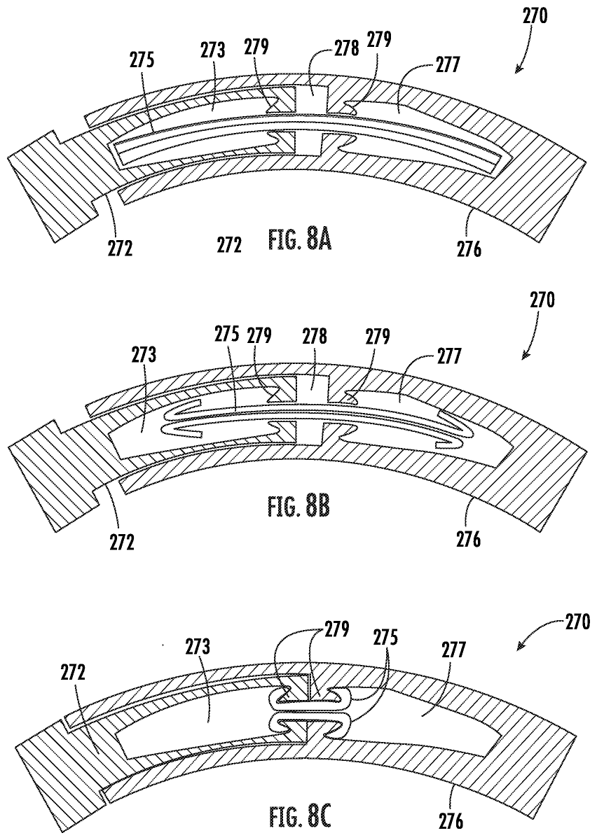

FIGS. 8A-8C show aspects of how the locking ring 270 can be secured within the external channel 245 of the elastic retainer 240. While the features shown in FIGS. 8A-8C are shown directed towards locking ring 270 of implant system 200, these features are also compatible with, and can be used to secure the ends of, the locking ring 170 of implant system 200 in FIGS. 1-3. As shown, the locking ring 270 has complementarily formed ends, the first end 272 of which nests within a recess 278 formed in the end second end 276. As can be seen in other figures, the locking ring 270 is a substantially continuous (e.g., between the first and second ends 272, 276) metal ring with a cross-sectional area of a circle, defining an annular path between the first and second ends 272, 276. As such, the first end 272 is at an opposite end of the locking ring 270 from the second end 276, but the first end 272 is connected to the second end 276 by the portion of the locking ring 270 that extends therebetween and defines the circular, or annular, shape of the locking ring 270 at least when the locking ring 270 is secured within the channel 245 of the elastic retainer 240.

As illustrated, FIGS. 8A-8C are cross-sectional images of a portion of the locking ring 270, showing various stages of engagement between the first and second ends 272, 276 of the locking ring 270. During the surgical implantation of the implant system 200, at least one Nitinol shape-memory wire 275 is inserted within, and extending between, cavities 273, 277 of the first and second ends 272, 276 of the locking ring 270. Nitinol is a generic for a material, in this case wire, comprised of nickel and titanium and having thermal-dependent shape memory characteristics. While the fasteners that secure the first and second ends 272, 276 of the locking ring 270 together are shown herein as advantageously being Nitinol wires 275, these can be replaced with any suitable structure and/or material that will secure the first and second ends 272, 276 of the locking ring 270 together. It is advantageous to provide a plurality of (e.g., two or more of) the Nitinol wires 275. Retention features 279, here in the shape of an angled flange, are provided at the apertures of each of the cavities 273, 277 and directed inwardly into each of the cavities 273, 277. Upon initial insertion of the first end 272 within the recess 278 of the second end 276, such that the first end 272 is not fully inserted within the recess 278 of the second end 276. This arrangement is shown in FIG. 8A, in which the locking ring 270 is located within, but is not secured within, the channel 245 of the elastic retainer 240.

The shape-memory aspects of the Nitinol wires 275 are thermal, or heat, dependent. As such, the Nitinol wires 275 have a substantially straight arrangement as shown in FIG. 8A, when at a first temperature. Upon heating of the Nitinol wires 275 beyond the first temperature, the shape of the Nitinol wires 275 start to change shape to transition towards the locked “C” shape shown in FIG. 8C, which occurs at or before a second temperature, the second temperature being greater than the first temperature and the second temperature being at the same or less than the internal body temperature of the subject in which the implant system 200 is to be implanted. As the temperature of the Nitinol wires 275 increases, the ends of the Nitinol wires 275 begin to curl, or form hooks, that can latch onto the retention features 279 within the cavities 273, 277 of the first and second ends 272, 276 of the locking ring 270. This intermediate stage is shown in FIG. 8B. As the temperature of the Nitinol wires 275 increases, the Nitinol wires 275 shorten in length and curl into a tighter and shorter “C” shape, as shown in FIG. 8C, thereby latching onto and/or over the retention features 279 within the cavities 273, 277 and pulling the first end 272 substantially fully within the recess 278 of the second end 276, thereby securing the locking ring 270 within the channel 245 of the elastic retainer 240.

In another example embodiment, a method of replacing a hip joint in a patient is provided. Such methods can include providing a patient and/or subject in need of surgical hip replacement, providing an implant system configured as a hip replacement implant, as disclosed herein in FIGS. 1-3, and surgically implanting the implant system into the patient. As used herein, the term “patient” is to be interpreted broadly, including not only a human subject, but any animal having joints in which the use of such implant systems as disclosed herein are suitable for use in joint replacement surgical procedures. Retention of the implant system can be substantially increased post surgery using such methods.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

1. An implant system for reconstructive surgery of a ball-and-socket joint, the implant system comprising:
a cup having a substantially hemispherical inner contour;
a head configured for at least partial insertion within the cup, the head having an outer contour that is spherically-shaped, at least over portions of the head that are capable of contacting the inner contour of the cup over a predetermined range of motion of the ball-and-socket joint; and
an elastic retainer comprising an inner contour that is at least a cross-sectional segment of a spherical shape having substantially a same radius as the inner contour of the cup, wherein the elastic retainer is configured for attachment over an open end of the cup to partially cover and retain the head within the cup, and wherein, when securing the head within the cup, the elastic retainer is configured to allow the outer contour of the head to freely rotate and/or pivot against the inner contour of the cup over the predetermined range of motion of the ball-and-socket joint;
wherein the elastic retainer is configured to prevent post-surgical dislocation of the head from the cup.

2. The implant system of claim 1, wherein the elastic retainer comprises an elastic material having an elasticity and tensile strength that is sufficient to provide dynamic support to the head and cup.

3. The implant system of claim 2, wherein the elastic retainer comprises a high-durometer elastomeric material.

4. The implant system of claim 1, wherein the inner contour of the elastic retainer forms a lip at an upper surface of the elastic retainer, the lip having a narrower diameter than a diameter of the head for retaining the head within the cup.

5. The implant system of claim 4, wherein the elastic component has an exterior channel formed circumferentially about and adjacent to the upper surface of the elastic retainer, wherein the exterior channel is configured to retain a locking ring therein.

6. The implant system of claim 5, wherein the locking ring is positioned within the exterior channel of the elastic retainer and is configured to resist radial expansion of the elastic retainer to resist a dislocation of the head from the cup.

7. The implant system of claim 6, wherein the locking ring comprises a first end and a second end, the second end having a recess formed therein, wherein the first end is configured for insertion within the recess of the second end to secure the first and second ends of the locking ring together in a substantially continuous annular shape.

8. The implant system of claim 7, wherein each of the first and second ends comprise an internal cavity, wherein one or more shape-memory wires is positioned to extend between and into the cavity of the first end and the cavity of the second end, and wherein the one or more shape-memory wires are configured to change a shape thereof based on a temperature of the one or more shape-memory wires.

9. The implant system of claim 8, wherein the one or more shape-memory wires comprise Nitinol.

10. The implant system of claim 8, wherein the one or more shape-memory wires are substantially straight at a first temperature, wherein a retention feature is formed within each cavity of the first and second ends, and wherein the one or more shape-memory wires are configured to engage with the retention features of each cavity at a second temperature to pull the first end of the locking ring within the second end of the locking ring to substantially entirely fill the recess of the second end of the locking ring.

11. The implant system of claim 1, wherein the inner contour of the elastic component comprises a low friction bearing surface.

12. The implant system of claim 1, wherein a lower surface of the elastic retainer is configured to be fixed coincidently and concentrically over an upper, open surface of the cup, the elastic retainer having a substantially similar diameter to a diameter of the cup at the upper, open surface of the cup.

13. The implant system of claim 1, wherein the cup comprises an inner liner that is concentrically attached within the cup, the inner liner comprising an upper annular flange extending from an upper surface of the cup, wherein the elastic retainer comprises an annular channel having a cross-sectional shape that is substantially similar to a cross-sectional shape of the upper annular flange, and wherein the upper annular flange of the inner liner is inserted within the annular channel of the elastic retainer to affix the elastic retainer to the cup.

14. The implant system of claim 13, wherein the elastic retainer is molded over the upper annular flange of the inner liner, such that the elastic retainer and the inner liner are of a substantially unitary, or monolithic, construction.

15. The implant system of claim 13, wherein the upper annular flange comprises holes formed through a thickness thereof in the radial direction, such that a material of the elastic retainer solidifies within the holes to secure the elastic retainer to the inner liner.

16. The implant system of claim 1, wherein the elastic retainer comprises holes in one or more sides of the elastic retainer that allow, when the head receives a force in a direction of dislocation, for the elastic retainer to be compressed and/or deformed by reducing a size of the holes, while maintaining sufficient tensile strength of the elastic retainer to retain the head within the cup.

17. The implant system of claim 1, wherein the head and/or cup comprise a metal or metal alloy.

18. A method of surgically reconstructing a ball-and-socket joint in a subject, the method comprising:
providing the subject in need of surgical reconstruction of the ball-and-socket joint;
providing an implant device for surgically reconstructing the ball-and-socket joint, the implant device comprising:
a cup having a substantially hemispherical inner contour;
a head having an outer contour that is spherically-shaped, at least over portions of the head that are capable of contacting the inner contour of the cup over a predetermined range of motion of the ball-and-socket joint when implanted within the patient; and
an elastic retainer comprising an inner contour that is at least a cross-sectional segment of a spherical shape having substantially a same radius as the inner contour of the cup;
attaching the cup to or within a first anatomical structure of the subject, the first anatomical structure corresponding to a socket portion of the ball-and-socket joint;
inserting the head, at least partially, within the cup;
attaching the elastic retainer over an open end of the cup to partially cover and retain the head within the cup, thereby securing the head within the cup, wherein the elastic retainer allows the outer contour of the head to freely rotate and/or pivot against the inner contour of the cup over the predetermined range of motion of the ball-and-socket joint;
attaching a stem to or within a second anatomical structure of the subject, the second anatomical structure corresponding to a ball portion of the ball-and-socket joint;
fixedly attaching the head to the stem, so that the head is rigidly attached to the stem to prevent relative movements between the head and the stem; and
resisting, when a force is received in a direction of dislocation, a dislocation of the head from the cup after surgery.

19. The method of claim 18, wherein:
the cup comprises an inner liner that is concentrically attached within the cup, the inner liner comprising an upper annular flange extending from an upper surface of the cup; and
the elastic retainer comprises an annular channel having a cross-sectional shape that is substantially similar to a cross-sectional shape of the upper annular flange;
the method comprising:
molding the elastic retainer over the upper annular flange of the inner liner, such that the annular channel of the elastic retainer is formed around the upper annular flange of the inner liner and the elastic retainer and the inner liner are of a substantially unitary, or monolithic, construction.

20. The method of claim 18, wherein the first anatomical structure is an acetabulum of a pelvis of the subject and the second anatomical structure is a femur of the subject.
